(19) United States
(12) Patent Application Publication
(10) Pub. No.: US 2018/0110658 A1
(43) Pub. Date: Apr. 26, 2018

(54) Thin Patch

(71) Applicant: Hsueh-Ya Lin, Kaohsiung City (TW)

(72) Inventor: Hsueh-Ya Lin, Kaohsiung City (TW)

(21) Appl. No.: 15/782,903

(22) Filed: Oct. 13, 2017

(30) Foreign Application Priority Data
Oct. 21, 2016 (TW) 105216141

(51) Int. Cl. A61F 13/02; A61K 9/70

(52) U.S. Cl. CPC A61F 13/024; A61K 9/7038; A61F 13/0253; A61F 13/0259

(57) ABSTRACT

Provided is a thin patch having a flexible patch body, an outer layer, and a release layer. The flexible patch body has a waterproof and moisture-permeable layer and an adhesive gel layer. The outer layer is attached on the waterproof and moisture-permeable layer. The outer layer has a left wrapper and a right wrapper which are adjacent to each other. The release layer includes a first release film and a second release film. The first release film includes a release part attached on a side of the adhesive gel layer and a connecting part extending from the release part of the first release film and folded away from the adhesive gel layer. The second release film is attached on a part of the adhesive gel layer and covers a part of the connecting part of the first release film.

### BACKGROUND OF THE INVENTION

### 1. Field of the Invention

The present invention relates to a patch, in particular to a thin skin patch for humans.

### 2. Description of the Prior Arts

When having stiff or sore muscles, people usually seek for a traditional way such as osteopathic manipulation, massage, acupuncture, or oral painkillers to relieve their discomfort. However, the first three ways take too much time and thus are not suitable for people with a busy, fast-paced lifestyle. Oral painkillers are a huge burden for the human body and therefore are not suitable for long-term administration. Due to the shortcomings described above, pain relief patches with medical or relieving substances in their dressings have recently become popular. By covering the patch on the skin, the active substances of the dressing would be absorbed, so as to ease the discomfort such as muscle sore or stiffness.

With the popularity of the use of pain relief patch, a variety of patches with different effects or smells are available on the market. Conventional patches are each composed of an adhesive layer and a protective layer at two opposite sides, and the adhesive layer is covered with a release film. When using it, the user peels off the release film and covers the patch onto the skin through the adhesive layer by hand. However, after peeling off the release film, the user's hand will directly contact the adhesive layer, resulting in the reduced adhesion of the patch due to repeated touch. The reduced adhesion not only causes the inconvenient operation, but also increases the possibility of the patch's detachment from the skin. Moreover, the thinner the patch, the higher the flexibility, and thus the adhesive layer of the thinner patch is more likely to cause inadvertent self-adhesion, making the patch unusable and wasting the patch.

### SUMMARY OF THE INVENTION

In view of the shortcomings of the prior art, the objective of this invention is to provide a thin patch that is easy-peeling, non-sticky to the hand, convenient in operation, and affixed to the skin.

To achieve the foresaid objective, the present invention provides a thin patch which comprises: a flexible patch body, an outer layer, and a release layer. The flexible patch body comprises a waterproof and moisture-permeable layer and an adhesive gel layer, and the adhesive gel layer is coated on a back side of the waterproof and moisture-permeable layer. The outer layer is removably attached on the waterproof and moisture-permeable layer of the flexible patch body. The outer layer comprises a left wrapper and a right wrapper, the left wrapper and the right wrapper are respectively and removably attached on two sides of the waterproof and moisture-permeable layer, and the left wrapper is spaced apart from the right wrapper. The release layer is removably attached on the adhesive gel layer of the flexible patch body, and the release layer comprises a first release film and a second release film. The first release film of the release layer is attached on the adhesive gel layer and comprises a release part attached on a side of the adhesive gel layer and a connecting part extending from the release part of the first release film and folded away from the adhesive gel layer. The second release film of the release layer is attached on a part of the adhesive gel layer and covers a part of the connecting part of the first release film.

Preferably, the adhesive gel layer comprises milk peptide, nanogold, glutamic acid fermentates, plant extracts, vitamin B complex, vitamin C, vitamin D3, pressure-sensitive adhesive, pure water or any mixtures thereof. Preferably, the plant extracts in the adhesive gel layer comprise glucosamine, bromelain, suanzaoren extracts, gastrodia extracts, transamin, curcumin, eucalyptus essential oil, rosemary essential oil, chamomile essential oil, or mint essential oil.

Preferably, an edge of the left wrapper that is away from the adhesive layer is complementary in a wave shape with an edge of the right wrapper that is away from the adhesive layer, and the left wrapper is spaced apart from the right wrapper on the waterproof and moisture-permeable layer.

Preferably, an area of the second release film is smaller than an area of the adhesive gel layer and larger than an area of the first release film. The second release film comprises an adhesive part and a stripping part, which extends from the adhesive part and is spaced apart from the release part of the first release film.

Preferably, each of the left wrapper and the right wrapper comprises an adhesive layer and an extension layer extending from the adhesive layer; i.e., the left wrapper comprises an adhesive layer and an extension layer extending from the adhesive layer of the left wrapper, and the right wrapper comprises an adhesive layer and an extension layer extending from the adhesive layer of the right wrapper. The adhesive layer of the left wrapper and the adhesive layer of the right wrapper are respectively and removably attached on two sides of the waterproof and moisture-permeable layer, the adhesive layer of the left wrapper is spaced apart from the adhesive layer of the right wrapper, and the extension layer of the left wrapper and the extension layer of the right wrapper are not adhered onto the waterproof and moisture-permeable layer. Preferably, an edge of the extension layer of the left wrapper that is away from the adhesive layer is complementary in a wave shape with an edge of the extension layer of the right wrapper that is away from the adhesive layer.

Preferably, a flexible patch body of the thin patch in accordance with the present invention is provided between the outer layer and the release layer. The release layer keeps the adhesive gel layer of the thin patch from being sticky and the outer layer strengthens the structure of the flexible patch body so that the thin patch will not be self-adhesive. The second release film of the release layer covers a part of the connecting part of the first release film so that the user can easily grasp the stripping part of the second release film to peel off the second release film from the adhesive gel layer. After a part of the adhesive gel layer is adhered onto the user's skin, the connecting part of the first release film is grasped to peel off the first release film from the adhesive gel layer of the flexible patch body, and then the adhesive gel layer is completely adhered to the user's skin without sticking to the user's hand. The user can then grasp the extension layer of the left wrapper and the extension of the right wrapper of the outer layer and peel off the left wrapper and the right wrapper from the opposite directions, making the patch affixed to the user's skin. Therefore, the present invention provides the patch that is easy to peel off, non-sticky to hands, convenient in operation, and affixed to the skin.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a thin patch in accordance with the present invention;

FIG. 2 is another perspective view of the thin patch in accordance with the present invention;

FIG. 3 is an exploded view of the thin patch in accordance with the present invention;

FIG. 4 is a side cross-sectional view of the thin patch in accordance with the present invention;

FIG. 5A is a partially enlarged view of the thin patch in accordance with the present invention; and

FIG. 5B is another partially enlarged view of the thin patch in accordance with the present invention.

### DETAILED DESCRIPTION OF THE PREFERRED EMBODIMENTS

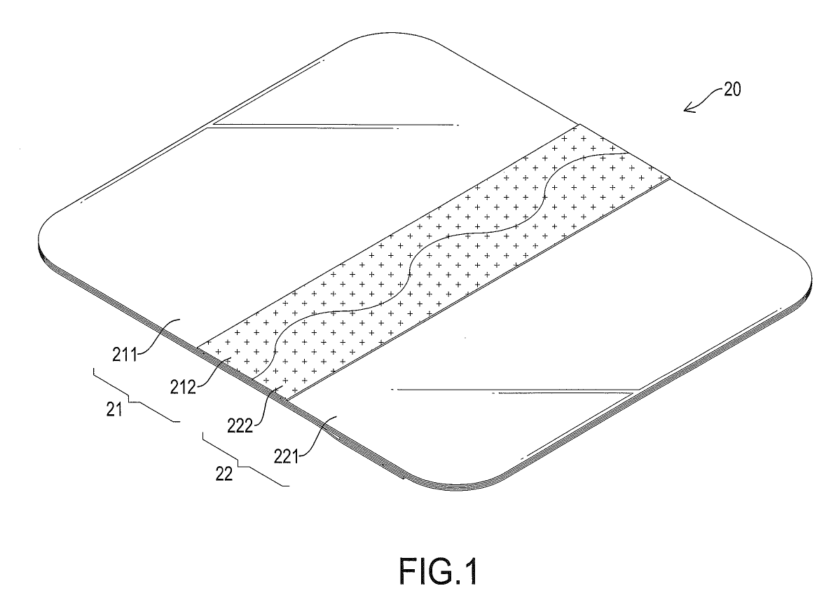

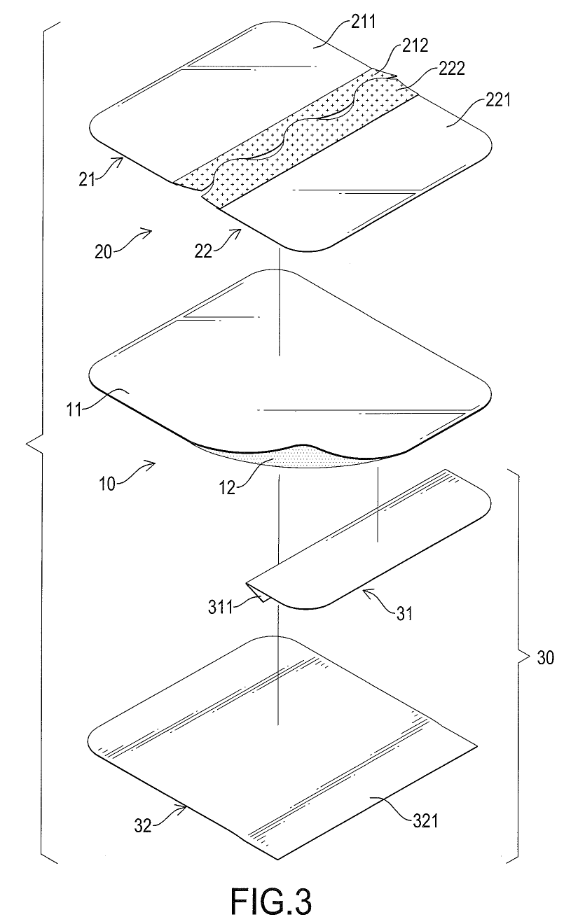

As illustrated in FIG. 1 to FIG. 3, a thin patch of the present invention comprises a flexible patch body 10, an outer layer 20, and a release layer 30. The flexible patch body 10 comprises a waterproof and moisture-permeable layer 11 and an adhesive gel layer 12. A shape of the adhesive gel layer 12 matches with a shape of the waterproof and moisture-permeable layer 11, and the adhesive gel layer 12 is coated on a back side of the waterproof and moisture-permeable layer 11. The adhesive gel layer 12 comprises milk peptide, nanogold, glutamic acid fermentates, glucosamine, bromelain, suanzaoren extracts, gastrodia extracts, transamin, curcumin, eucalyptus essential oil, rosemary essential oil, chamomile essential oil, mint essential oil, vitamin B complex, vitamin C, vitamin D3, pressure-sensitive adhesive, pure water or any mixtures thereof. The adhesive gel layer 12 can be made of one of the following five mixtures, mixture 1: curcumin, eucalyptus essential oil, pressure-sensitive adhesive, and water; mixture 2: milk peptide, glutamic acid fermentates, suanzaoren extracts, vitamin B complex, gastrodia extracts, and pressure-sensitive adhesive; mixture 3: rosemary essential oil, chamomile essential oil, mint essential oil, pressure-sensitive adhesive, and pure water; mixture 4: transamin, vitamin C, pressure-sensitive adhesive, and pure water; and mixture 5: glucosamine, bromelain, nanogold, vitamin C, pressure-sensitive adhesive, and pure water.

As illustrated in FIG. 1 to FIG. 3, the outer layer 20 is removably attached on the waterproof and moisture-permeable layer 11 of the flexible patch body 10, and the outer layer 20 comprises: a left wrapper 21 and a right wrapper 22, each wrapper comprising a respective adhesive layer 211, 221 and a respective extension layer 212, 222 extending from the adhesive layer 211, 221. The adhesive layer 211 of the left wrapper 21 and the adhesive layer 221 of the right wrapper 22 are respectively and removably attached on two opposite sides of the waterproof and moisture-permeable layer 11. The adhesive layer 211 of the left wrapper 21 is spaced apart from the adhesive layer 221 of the right wrapper 22, and the extension layer 212 of the left wrapper 21 and the extension layer 222 of the right wrapper 22 are not adhered onto the waterproof and moisture-permeable layer 11. An edge of the extension layer 212 of the left wrapper 21 that is away from the adhesive layer 211 is complementary in a wave shape with an edge of the extension layer 222 of the right wrapper 22 that is away from the adhesive layer 221.

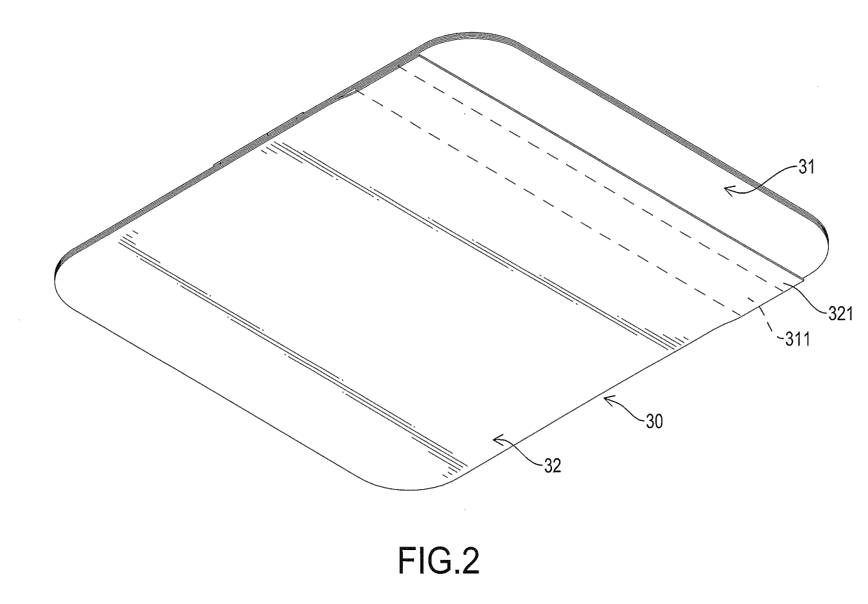

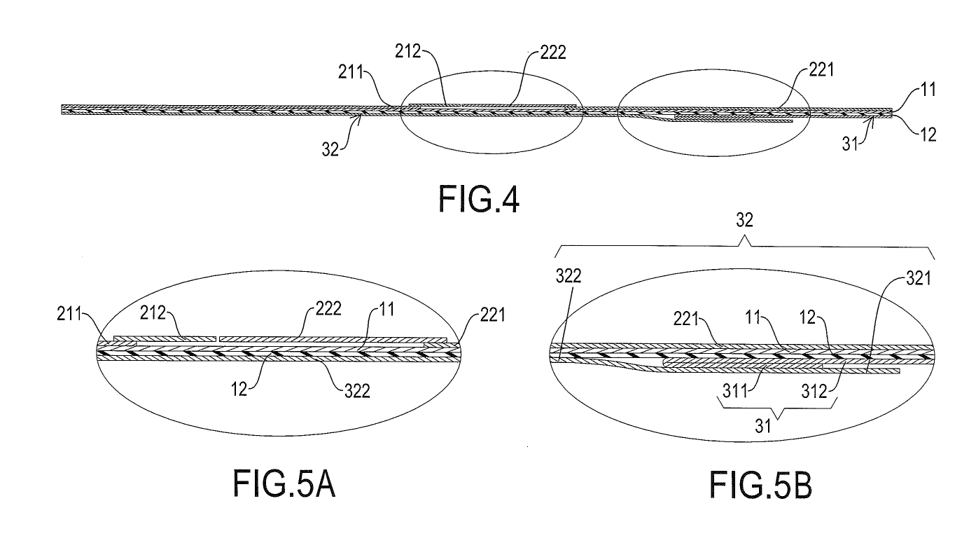

As illustrated in FIGS. 2 to 4, the release layer 30 is removably attached on a side of the adhesive gel layer 12 of the flexible patch body 10 and is spaced apart from the waterproof and moisture-permeable layer 11. The release layer 30 comprises a first release film 31 and a second release film 32. The first release film 31 comprises a release part 312 attached on a side of the adhesive gel layer 12 and a connecting part 311 extending from the release part 312 of the first release film 31 and is folded away from the adhesive gel layer 12. The second release film 32 is attached on a part of the adhesive gel layer 12 and covers a part of the connecting part 311 of the first release film 31. The second release film 32 comprises an adhesive part 322 and a stripping part 321 extending from the adhesive part 322 of the second release film 32 and is spaced apart from the release part 312 of the first release film 31.

In a best mode of the thin patch of the present invention, the user grasps the stripping part 321 of the second release film 32 of the release layer 30 to detach the second release film 32 from the adhesive gel layer 12 of the flexible patch body 10. Then, the user grasps the first release film 31 and adheres a part of the adhesive gel layer 12 to the user's skin to substantially attach the flexible patch body 10 onto the skin. After that, the user grasps the release part 311 of the first release film 31 to detach the first release film 31 from the adhesive gel layer 12 of the flexible patch body 10, and the adhesive gel layer 12 of the flexible patch body 10 is completely adhered to the user's skin. The user can then grasp the extension layer 212 of the left wrapper 21 of the outer layer 20 and the extension layer 222 of the right wrapper 22 of the outer layer 20 and peel off the left wrapper 21 and the right wrapper 22 from the opposite directions. That is, the user grasps both the extension layer 212 of the left wrapper 21 and the extension layer 222 of the right wrapper 22 and peels off the adhesive layers 211 of the left wrapper 21 and the adhesive layer 221 of the right wrapper 22. To expose the waterproof and moisture-permeable layer 11 of the flexible patch body 10, the left wrapper 21 and the right wrapper 22 from the waterproof and moisture-permeable layer 11 are detached. The flexible patch body 10 is then affixed to the user's skin.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A patch, comprising:
a flexible patch body, the flexible patch body comprising:
a waterproof and moisture-permeable layer; and
an adhesive gel layer coated on a back side of the waterproof and moisture-permeable layer, a shape of the adhesive gel layer matching with a shape of the waterproof and moisture-permeable layer; and
an outer layer removably attached on the waterproof and moisture-permeable layer of the flexible patch body, the outer layer comprising:
a left wrapper; and
a right wrapper being adjacent to the left wrapper; and
a release layer removably attached on the adhesive gel layer of the flexible patch body and comprising:
a first release film attached on the adhesive gel layer and comprising:
a release part attached on a side of the adhesive gel layer; and
a connecting part extending from the release part of the first release film and folded away from the adhesive gel layer; and
a second release film attached on a part of the adhesive gel layer and covering a part of the connecting part of the first release film, and comprising:
an adhesive part attached on a part of the adhesive gel layer; and
a stripping part extending from the adhesive part of the second release film.

2. The patch as claimed in claim 1, wherein the adhesive gel layer comprises glutamic acid fermentates, glucosamine, bromelain, suanzaoren extracts, gastrodia extracts, transamin, curcumin, eucalyptus essential oil, rosemary essential oil, chamomile essential oil, or mint essential oil.

3. The patch as claimed in claim 2, wherein the adhesive gel layer comprises milk peptide, nanogold, vitamin B complex, vitamin C, vitamin D3, pressure-sensitive adhesive, pure water, or any mixtures thereof.

4. The patch as claimed in claim 1, wherein an edge of the right wrapper and an edge of the left wrapper are complementary to each other in a wave shape.

5. The patch as claimed in claim 1, wherein an area of the second release film is smaller than an area of the adhesive gel layer and larger than an area of the first release film.

6. The patch as claimed in claim 1, wherein the second release film comprises a stripping part extending from the adhesive part, and the stripping part is spaced apart from the release part of the first release film.

7. The patch as claimed in claim 1, wherein each of the left wrapper and the right wrapper comprises an adhesive layer and an extension layer extending from the adhesive layer; the adhesive layer of the left wrapper and the adhesive layer of the right wrapper are respectively and removably attached on two sides of the waterproof and moisture-permeable layer, the adhesive layer of the left wrapper is spaced apart from the adhesive layer of the right wrapper, and the extension layer of the left wrapper and the extension layer of the right wrapper are not adhered onto the waterproof and moisture-permeable layer.

8. The patch as claimed in claim 7, wherein an edge of the extension layer of the left wrapper that is away from the adhesive layer is complementary in a wave shape with an edge of the extension layer of the right wrapper that is away from adhesive layer.
